(19) United States
(12) Patent Application Publication
(10) Pub. No.: US 2006/0122701 A1
(43) Pub. Date: Jun. 8, 2006

(54) Posterior lumbar interbody fusion expandable cage with lordosis and method of deploying the same

(76) Inventor: P. Douglas Kiester, Irvine, CA (US)

Correspondence Address: Daniel L. Dawes;Myers Dawes Andras & Sherman LLP, Suite 1150, 19900 MacArthur Boulevard, Irvine, CA, 92612, US

(21) Appl. No.: 11/285,693

(22) Filed: Nov. 21, 2005

Related U.S. Application Data
(60) Provisional application No. 60/630,944, filed on Nov. 23, 2004.
(60) Provisional application No. 60/680,264, filed on May 11, 2005.

(51) Int. Cl. A61F 2/44; A61B 17/88

(52) U.S. Cl. 623/17.11; 606/90

(57) ABSTRACT

A spinal fusion cage comprises an upper half-cage, a lower half-cage, and a plunger with a cam. The upper half-cage and lower half-cage have a first collapsed configuration which has a thin, flat, rectangular envelope and a second expanded configuration. The half-cages have at least one ramped surface on which the cam of the plunger rides. The cam bears against the ramped surface and spreading the two half-cages apart. A method of deploying a spinal fusion cage comprises the steps of disposing in a spinal space an upper half-cage and lower half-cage in a first collapsed configuration which has a thin, flat, rectangular envelope and a second expanded configuration. The method continues with the step of distally advancing a plunger between the upper half-cage and lower half-cage and spreading the two half-cages apart.

### RELATED APPLICATIONS

The present application is related to U.S. Provisional Patent Application serial No. 60/630,944, filed on Nov. 23, 2004 and U.S. Provisional Patent Application serial No. 60/680,264, filed on May 11, 2005, which are incorporated herein by reference and to which priority is claimed pursuant to 35 USC 119.

### BACKGROUND OF THE INVENTION

1. Field of the Invention

The invention relates to the field of implantable fusion cages for use in the spinal column.

2. Description of the Prior Art

Fusion cages provide a space for inserting a bone graft between adjacent portions of bone. In time, the bone and bone graft grow together through or around the fusion cage to fuse the graft and the bone solidly together. One current use of fusion cages is to treat a variety of spinal disorders, including degenerative disc diseases, Grade I or II spondylolistheses, adult coliosis and other disorders of the lumbar spine. Spinal fusion cages (included in the general term, “fusion cages”) are inserted into the intervertebral disc space between two vertebrae for fusing them together. They distract (or expand) a collapsed disc space between two vertebrae to stabilize the vertebrae by preventing them from moving relative to each other.

The typical fusion cage is cylindrical, hollow, and threaded. Alternatively, some known fusion cages are unthreaded or made in tapered, elliptical, or rectangular shapes. Known fusion cages are constructed from a variety of materials including titanium alloys, porous tantalum, other metals, allograft bone, carbon fiber or ceramic material.

Fusion cages may be used to connect any adjacent portions of bone, however one primary use is in the lumbar spine. Fusion cages can also be used in the cervical or thoracic spine. Fusion cages can be inserted in the lumbar spine using an anterior, posterior, or lateral approach. Insertion is usually accomplished through a traditional open operation, but a laparoscopic or percutaneous insertion technique can also be used.

With any of the approaches, threaded fusion cages are inserted by first opening the disc space between two vertebrae of the lumbar spine using a wedge or other device on a first side of the vertebrae. Next, a tapered plug is hammered in to hold the disc space open in the case of a threaded, cylindrical cage insert. A threaded opening is then drilled and tapped on a second side opposite the first side of the vertebrae for producing the equivalent of a “split” threaded bore defined by the walls of the vertebrae above and below the bore. The threaded fusion cage is then threaded into the bore and the wedge is removed. The first side is then drilled and tapped before inserting a second threaded fusion cage. Typically, two threaded fusion cages are used at each invertebral disc level.

There are problems with all of the standard approaches. With a posterior approach, neural structures in the spinal canal and foramen need to be properly retracted before the plug is hammered or threaded into the disc space. Proper neural retraction is critical to the insertion process. If the retraction is not done properly, the procedure could cause neural injury, i.e., nerve damage and potential neurologic deficit. With either the anterior or lateral approach, blood vessels or other vital structures need to be retracted and protected to reduce or eliminate internal bleeding. Violation of the great vessels has a high mortality rate.

The general technique for inserting fusion cages is well known. Insertion techniques and additional details on the design of fusion cages is described in Internal Fixation and Fusion of the Lumbar Spine Using Threaded Interbody Cages, by Curtis A. Dickman, M. D., published in BNI Quarterly, Volume 13, No. 3, 1997, which is hereby incorporated by reference.

U.S. Pat. No. 5,782,832 to Larsen et al. (the “Larsen reference”) discloses an alternate type of spinal fusion implant. FIG. 1 of the Larsen reference shows an implant apparatus with two separable support components which are adapted for adjusting sliding movement relative to each other to selectively vary the overall width of the implant to accommodate vertebral columns of various sizes or to vary the supporting capacity of the implant during healing. Each of the support components include upper and lower plate portions that are operatively connected by respective linkage mechanisms. The linkage mechanisms allow relative movement of the upper and lower plate portions between an extended position and a collapsed position. The device disclosed in the Larsen reference has several problems. One problem is that, because the width of the implant is adjusted prior to insertion, a wide insertion slot is necessary despite the reduced profile presented by the collapsed implant. Another problem is that at least part of the linkage mechanism extends beyond the upper and lower plate portions, thus requiring more invasion into the body cavity to position the implant. Yet another problem is that the linkage mechanisms must be locked into the expanded position by conventional arrangements such as locking screws.

Brett, U.S. Pat. No. 6,126,689 (2000), illustrates an expandable and collapsible fusion cage, but it design is extremely complex and therefore expensive to manufacture and prone to failure in the field. Moreover, its complex linkages require special surgical skills in its deployment. Indeed, there is no reliable deployment mechanism. The Brett design requires large hinges which make it too large and therefore unsuitable for posterior insertion.

Within the past several years there has been a dramatic resurgence of interest in interbody lumbar spinal fusions without disruption of the vertebral body endplate. Part of this renewed direction has been due to waning popularity in both anterior and posterior approach cylindrical cage fusions. Interbody fusion seems to be more reliable than the classic posterior lateral fusion for several reasons. First, the two endplates of the vertebral bodies are close together, and under compression toward each other. Second, there is a large surface area to fuse. Visualization of the nerve roots is easily done from any posterior approach.

Shortcomings have included difficulty getting lumbar lordosis, and placing a large graft through a small hole. Trans-facet lateral fusion has recently been introduced to overcome the small hole problem. In this procedure the entire facet is removed making a much wider access to the anterior disk space. To combat the instability problems this would cause the procedure is usually done only from one side, and almost always combined with pedicle screws.

A major reason to further develop good posterior approach fusions is that it avoids the anterior surgical approach with all of its inherent risks. Indeed, it is the low but real incidence of major complications associated with the anterior surgical approach which is largely responsible for the decreasing popularity of anteriorly placed cylindrical cages.

Kiester, U.S. Pat. No. 6,893,464 (2005) provided for an improved design, but incorporated a design which was not as strong or rugged as might be desired in some applications.

The prior art designs for posterior lumbar interbody fusion expandable cages all suffer from the common defect that they interfere with spinal fusion. What is needed is some type of posterior lumbar interbody fusion expandable cage which does not interfere with fusion, which is easier to manufacture, which is stronger and more reliable than the prior designs.

### BRIEF SUMMARY OF THE INVENTION

The illustrated embodiment of the invention is a spinal fusion cage comprising an upper half-cage, a lower half-cage, and a plunger with a cam. The upper half-cage and lower half-cage have a first collapsed configuration which has a thin, flat, rectangular envelope and a second expanded configuration. The half-cages have at least one ramped surface on which the cam of the plunger rides. The cam bears against the ramped surface and spreading the two half-cages apart.

The plunger has two cams and each half-cage has a ramped surface on which the cam of the plunger rides. The cam bears against the ramped surface and spreads the two half-cages apart by a predetermined distance proximally and distally to define a predetermined lordosis.

The plunger and half-cages each have an open structure to allow tissue infiltration therein.

The plunger and half-cages further comprise a locking mechanism so that when the plunger is fully inserted between the half-cages, the plunger is locked into position.

The locking mechanism locks the plunger between the half-cages at both the proximal and distal ends.

The upper and lower half-cages have an identical shape and are arranged and configured to mesh with each other in the collapsed configuration.

The half-cages each have a plurality of proximal ramps and a plurality of distal ramps. The plunger has a plurality of proximal cam surfaces arranged and configured to slide on the plurality of proximal ramps and a plurality of distal cam surfaces arranged and configured to slide on the plurality of distal ramps to spread the half-cages apart.

The plunger and half-cages are arranged and configured so that the operational combination of proximal cam surfaces with the proximal ramps and the operational combination of distal cam surfaces and distal ramps to spread the half-cages apart are inversely symmetric with respect to the longitudinal axis of the fusion cage.

The plunger and half-cages are arranged and configured so that the inversely symmetric operational combination of the cam surfaces and ramps with respect to the longitudinal axis of the fusion cage provide during assembly of the fusion cage use the corresponding surfaces on the left side of the upper half-cage with the right side of lower half-cage, and the right side of upper half-cage with the left side of lower half-cage.

The proximal end of the plunger has a defined first height so that the corresponding proximal ends of the half-cages when the fusion cage is fully assembled are separated by the distance determined by the first height of the proximal end of the plunger. The distal end of the plunger has a defined second height so that the corresponding distal ends of the half-cages when the fusion cage is fully assembled are separated by the distance determined by the second height of the distal end of the plunger to provide a predetermined degree of lordosis.

The half-cages further comprise flanges with notches defined therein. The plunger is initially coupled to the proximal end of the two half-cages by means of engagement of the cam on the plunger with the notches in the flanges defined in the half-cages, when the plunger longitudinally extends from the two half-cages in the proximal direction.

Thus, the illustrated embodiment can be alternatively described as a spinal fusion cage is comprised of an upper half-cage, a lower half-cage and a plunger, where the upper half-cage has a distal portion which nests with the opposing distal portion of the lower half-cage so that the two half-cages provide a thin, flat, rectangular envelope, the half-cages have at least one or two inner ramped surface on which a cam of the plunger rides, the cam bearing against the ramped surface and distally spreading the two half-cages apart, at least one ramped surface on the proximal portion of the plunger bearing against an adjacent proximal interior surface of at least one or two of the half-cages so that at the same time as the plunger is being slid distally, the half-cages are being forced apart proximally.

The illustrated embodiment is also characterized as an assembly kit for a spinal fusion cage is comprised of a removable pusher tool; an upper and lower half-cage having parallel longitudinal axes, and a plunger which is slid between the half-cages by means of the removable pusher tool, where the half-cages are shaped in a complementary fashion so that when the upper half-cage is disposed on top of the lower half-cage, a distal portion of the lower and upper half-cages mesh with each other, so that the two half-cages assume the form of a collapsed fusion cage to provide a thin, flat, rectangular envelope with parallel upper and lower surfaces, the half-cages have opposing inner ramped surfaces on which a top and bottom cam surface of the plunger rides, as the plunger is forced distally between the two half-cages by the pusher tool, the cam which bears against the ramped surfaces on the interior surfaces of the two half-cages distally spreading the two half-cages apart in a direction perpendicular to their longitudinal axis, two symmetrically formed ramped surfaces on the proximal portion of the plunger bear against the proximal interior surfaces of the half-cages so that at the same time as the plunger is being slid distally towards a locked final position by the pusher tool, the half-cages are forced apart proximally, the ramps on the two half-cages and on the plunger being designed so that a desired degree of lordosis is obtained between the two-cages when in their final configuration.

The ramped interior surfaces have a mating and locking notch in the distal ends of the ramped interior surfaces of the two half-cages. The plunger has a cam which moves into the mating and locking notch when the cam reaches the end of the ramped interior surfaces of the two half-cages, the half-cages then moving together to capture and lock the plunger between them.

The half-cages have flanges with a proximal notch defined therein, where the plunger has a distal cam and is initially coupled to the proximal end of the two half-cages by means of engagement of the distal cam on the plunger is fitted into the notches in the flanges defined in the half-cages, while the plunger longitudinally extends from the two half-cages in the proximal direction.

The invention is further characterized as a method of deploying a spinal fusion cage comprising the steps of disposing in a spinal space an upper half-cage and lower half-cage in a first collapsed configuration which has a thin, flat, rectangular envelope and a second expanded configuration. The half-cages have at least one ramped surface on which the cam of the plunger rides. The method continues with the step of distally advancing a plunger between the upper half-cage and lower half-cage. The plunger has a cam bearing against the ramped surface and spreading the two half-cages apart.

The step of distally advancing the plunger comprises advancing a plunger with two cams and where each half-cage has a ramped surface on which the cam of the plunger rides, the cam bearing against the ramped surface and spreading the two half-cages apart by a predetermined distance proximally and distally to define a predetermined lordosis.

The method further comprises the step of providing the plunger and half-cages each with an open structure to allow tissue infiltration therein.

The method further comprises locking the plunger into position between the half-cages when the plunger is fully inserted between the half-cages.

The step of locking the plunger comprises locking the plunger between the half-cages at both the proximal and distal ends of the half-cages.

The step of disposing the upper half-cage and lower half-cage in a first collapsed configuration comprises disposing identically shaped upper and lower half-cages in an intermeshed relationship with each other in the collapsed configuration.

The step of distally advancing the plunger comprises sliding a plurality of proximal cam surfaces on the plunger on a plurality of proximal ramps and sliding a plurality of distal cam surfaces on the plurality of distal ramps to spread the half-cages apart.

The step of sliding a plurality of proximal cam surfaces on the plunger on a plurality of proximal ramps and sliding a plurality of distal cam surfaces on the plurality of distal ramps to spread the half-cages apart comprises operationally combining the proximal cam surfaces with the proximal ramps and the distal cam surfaces with distal ramps in a manner inversely symmetric with respect to the longitudinal axis of the fusion cage.

The step of operationally combining the proximal cam surfaces with the proximal ramps and the distal cam surfaces with distal ramps in a manner inversely symmetric with respect to the longitudinal axis of the fusion cage comprises assembling the fusion cage by using the corresponding surfaces on the left side of the upper half-cage with the right side of lower half-cage, and the right side of upper half-cage with the left side of lower half-cage.

The step of distally advancing the plunger comprises spreading the proximal ends of the half-cages by inserting therebetween a proximal end of the plunger which has a defined first height, and spreading the distal ends of the half-cages when the fusion cage by inserting therebetween the distal end of the plunger which has a defined second height so that when the fusion cage is fully assembled a predetermined degree of lordosis is provided.

The method further comprises the step of initially coupling the plunger into proximal notches defined in flanges of the half-cages by means of engagement of the cam on the plunger, when the plunger longitudinally extends from the two half-cages in a proximal direction.

The method further comprises the step of coupling a removable pusher tool to the plunger prior to distally advancing the plunger, automatically disengaging the pusher tool from the plunger after the plunger has been distally advanced beyond a predetermined distance between the half-cages, locking the plunger to the half-cages by distally advancing the tool, and removing the pusher tool.

The step of locking the plunger comprises engaging a cam on the plunger with a mating and locking notch in the distal ends of ramped interior surfaces of the two half-cages.

The step of locking the plunger comprises engaging the plunger with a mating and locking notch in the proximal ends of the two half-cages.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a perspective view of the fusion cage of the invention in a first configuration ready for implantation and wherein the plunger is coupled to the pusher tool which is resiliently connected to the plunger by a pair of opposing leaf springs.

FIG. 2 is a perspective view of the fusion cage of the invention as the pusher tool begins to push the plunger between the two half-cages, forcing the distal cam of the plunger between the opposing interior ramped surfaces of the two, nested, half-cages to spread them apart.

FIG. 3 is a perspective view of the fusion cage of the invention as the pusher tool pushed the plunger between the two half-cages, forcing the proximal portions of the two half-cages apart by means of contact with two opposing interior proximal ramped surfaces of the plunger to spread the proximal portion of the half-cages apart.

FIG. 4 is a perspective view of the fusion cage of the invention in which the pusher has forced the plunger into its forward most locked position wherein the two-half-cages are in an expanded and locked configuration.

FIG. 5 is a perspective view of the two assembled half-cages of the invention with the pusher tool being withdrawn after the plunger has been fully inserted and locked into place.

FIG. 6 is a side perspective view of the plunger.

FIG. 7 is a bottom perspective view of the half-cage.

FIG. 8 is a top perspective view of the half-cage. Both half-cages have the same shape and mesh with each other in the view of FIG. 1.

FIG. 9 is a proximal perspective end view of the plunger showing its left/right mirrored symmetry of shape.

FIG. 10 is a proximal perspective top view of the half-cage showing the proximal notches more clearly.

FIG. 11 is a bottom perspective view of the half-cage turned from the view of FIG. 7 to more clearly show the bottom surfaces of the half-cage which are hidden from view in FIG. 7.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

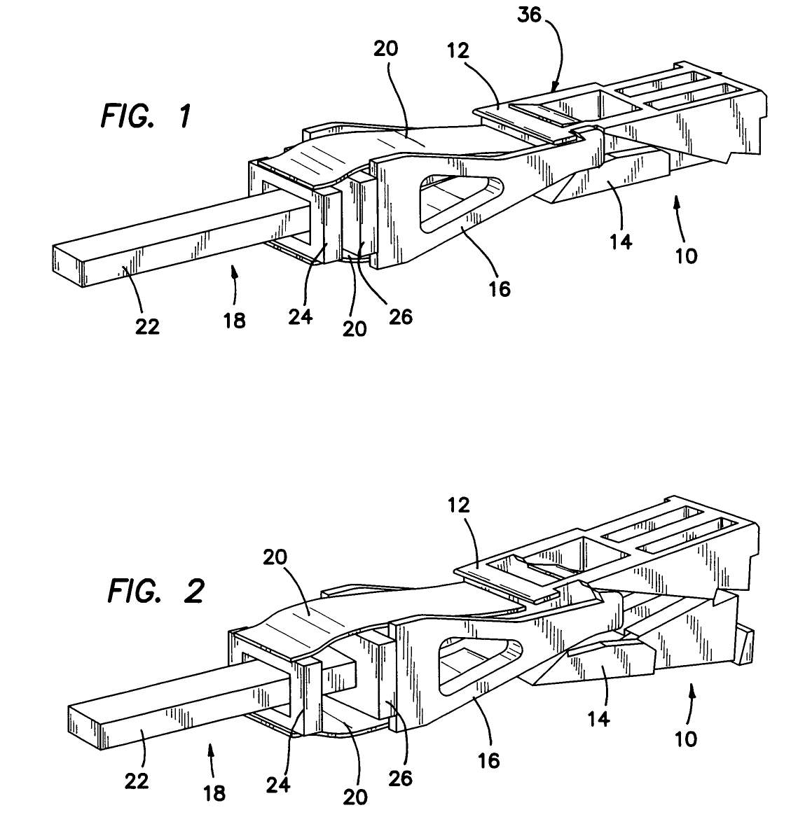

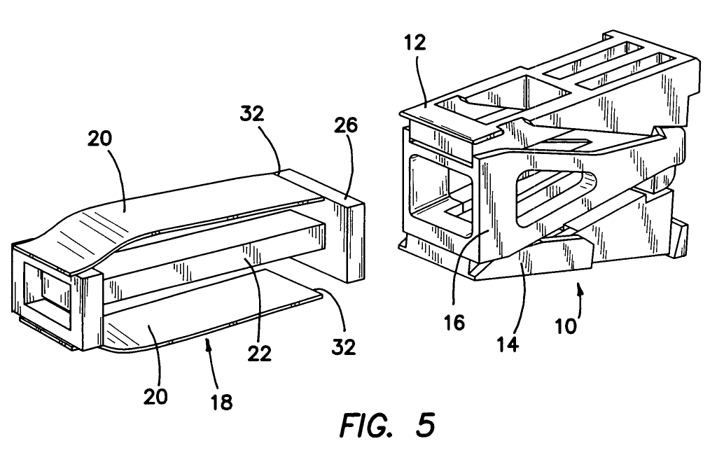

The spinal fusion cage 10 as best shown in assembled perspective view of FIG. 5 is comprised of two half-cages 12 and 14 and a middle plunger 16 which is slid between the half-cages 12 and 14 by means of a removable pusher tool 18. It should be borne in mind that during the sequence of steps illustrated in FIGS. 1-6 that half-cages 12 and 14 will be inserted into the spinal column between two adjacent vertebrae and hence will tend to pushed together by the vertebrae. The assembly of fusion cage 10 is then done against the opposing force of the adjacent vertebrae, which will be fused together in a predetermined position with a predetermined relative angle between them as provided by the invention.

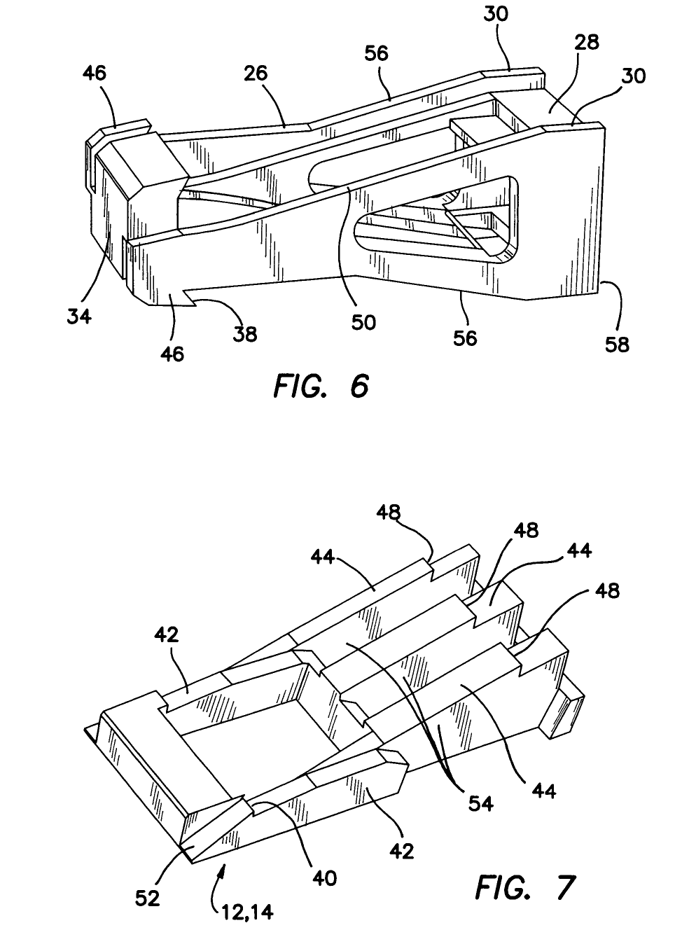

Turning first to the unassembled perspective view of FIG. 1 we see that the pusher tool 18 is coupled to the plunger 16 by two opposing leaf springs 20 that clasp opposing top and bottom sides of the plunger 16 and slide off the plunger 16 as it is forced between the two half-cages 12 and 14 as illustrated in the sequence of drawings of FIGS. 2-4. Tool 18 is comprised of a pusher rod 22 which may be of any length desired and coupled to or modified as appropriate to be coupled to a manipulation handle or any other tool manually used by the surgeon to manipulate the fusion cage 10 as it is being placed into the spine and assembled as taught by the invention. Rod 22 is disposed through a hole in a collar 24 to which springs 20 are coupled. The end of rod 22 is comprised of a flat pusher plate 26 to provide an even and stable force to be applied through rod 22 to the left end of plunger 16 as illustrated in the FIG. 1 Springs 20 extend across and resiliently clasp plunger 16 by means of a biased contact against surface 28 lying between flanges 30. The distal ends of springs 20 may extend down opposing raceways 26 defined on the interior longitudinal surface of plunger 16. Plunger 16 is preferably a molded or cast element composed of a biocompatible material, such as stainless steel or ceramic, but may be machined if desired. The distal ends 32 of springs 20 as best seen in FIG. 5 will in the configuration of FIGS. 1-4 either fall short of the adjacent edge of half-cages 12 and 14 or be biased to lie beneath them. In either case, the distal ends 32 of springs 20 are arranged and configure so as not to interfere with the insertion of the distal end 34 of plunger 16 as best seen in FIG. 6. The preferred configuration of rod 22, springs 20 and plate 26 is shown in FIG. 5, which is the configuration achieved when rod 22 has been extended to its maximum extent to completely insert plunger 16 between half-cages 12 and 14 and come to rest in a locked position as best shown in FIG. 4 and after it has been pulled back from assembled fusion cage 10.

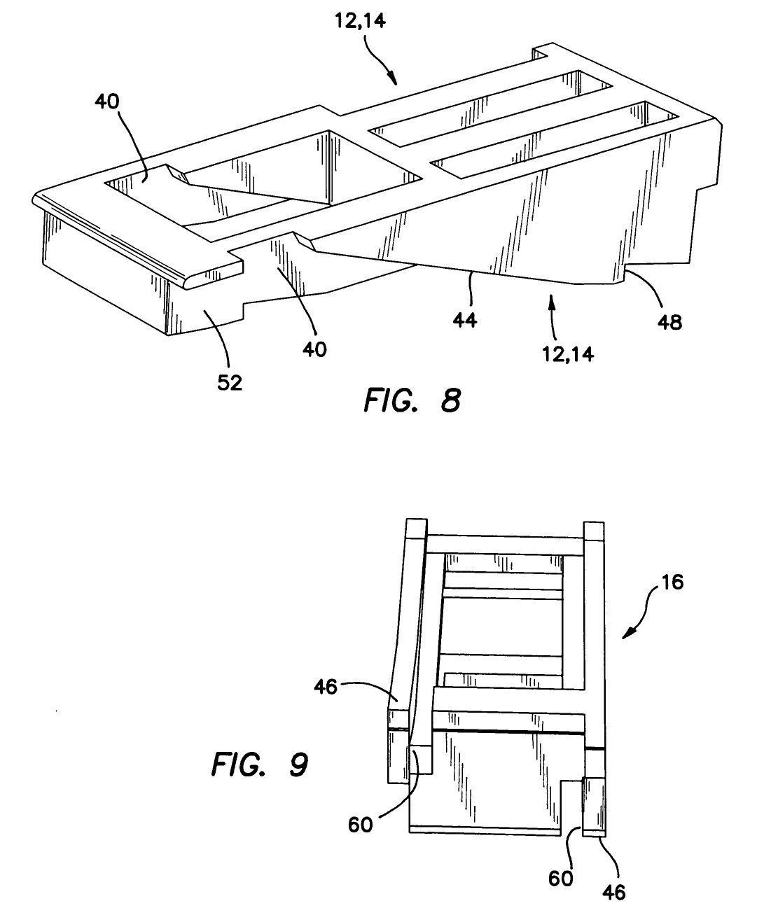

The two half-cages 12 and 14 are shaped in a complementary fashion so that the upper half-cage 12 is on top of and its distal portion meshes with the opposing distal portion of the lower half-cage 14, i.e. the distal portions of the upper and lower half-cages 12 and 14 are initially nested or meshed within each other so that the two half-cages 12 and 14 assume the form of a collapsed fusion cage and provide a thin, flat, rectangular envelope with parallel upper and lower surfaces 36 as best shown in the configuration of FIG. 1. Thus, it can be seen in FIG. 7 that surfaces 44 are offset from the longitudinal axis of the half-cage so that when flipped the opposed flanges providing surfaces 44 will mesh or nest with other. In this manner the two half-cages 12 and 14 can easily be surgical slid between two adjacent vertebrae. Greater detail of the shape of half-cages 12 and 14 are shown in the bottom and top perspective views of FIGS. 7 and 8. More of the detail of the shape and surfaces of half-cages 12 and 14 is described below in the context of assembly of fusion cage 10.

The plunger 16 is initially coupled to the proximal end of the two half-cages 12 and 14 by means of engagement as shown in FIG. 1 of a distal cam 38 best shown in FIG. 6 on the plunger 16 which fits into notches 40 defined in the two half-cages 12 and 14 as best seen in FIG. 8. This coupling allows plunger 16 which is grasped by springs 20 of tool 18 to handled as an integral unit and to be inserted and withdrawn from the surgical site. Initially the plunger 16 longitudinally extends from the two half-cages 12 and 14 in the proximal direction as depicted in FIG. 1. The two half-cages 12 and 14 have opposing inner ramped surfaces 44 seen in FIGS. 7 and 8 on which rides a top and bottom cam surface 46 seen in FIGS. 6 and 9 of the plunger 16. As the plunger 16 is forced distally between the two half-cages 12 and 14, the cams 46 that bear against the interior ramped surfaces 44 of the two half-cages 12 and 14, spreads half-cages 12 and 14 apart in a direction perpendicular to the longitudinal axis of fusion cage 10 as depicted in the sequence of steps of FIGS. 2-4.

FIG. 9 is a perspective proximal end view of plunger 16 in which slots 60 have been provided through plunger 16 to allow for nested fitting of plunger 16 with corresponding flanges 54 of half-cages 12 and 14 when plunger 16 is inserted between the two interior surfaces of half-cages 12 and 14.

When the cam 46 of the plunger 16 reaches the distal end of the ramped interior surfaces 44 of the two half-cages 12 and 14, cams 46 move into a mating and locking notches 48 seen in FIGS. 7 and 8 in the distal ends of the ramped interior surfaces 44 of the two half-cages 12 and 14. The two half-cages 12 and 14 then snap together under the forcing pressure of the adjacent vertebrae capturing and locking the plunger 16 between them to assume the assembled configuration of FIG. 5. The result is an expanded fusion cage 10 of FIG. 5.

At the same time as the plunger 16 is being slid distally towards its locked final position shown in FIG. 5, two symmetrically formed ramped surfaces 50 on the proximal portion of the plunger 16 as seen in FIG. 6 bear against the proximal interior surfaces 42 as seen in FIG. 11 of the two half-cages 12 and 14, forcing them apart. The ramped surfaces 46 on the two half-cages 12 and 14 and on the plunger 16 are designed so that desired degree of lordosis is obtained between the two half-cages 12 and 14 when in the final locked position of FIG. 5. The proximal end of half-cages 12 and 14 can thus be seen to nest inside of proximal flanges 30 of plunger 16 when cage 10 is fully assembled. Proximal surfaces 52 of half-cages 12 and 14 ride along surfaces 26 of plunger 16. Meanwhile distal cams 46 of plunger 16 ride on surfaces 44 of half-cages 12 and 14. In this manner there is an expansive force being simultaneously applied to force the two half-cages 12 and 14 apart both at their proximal and distal ends.

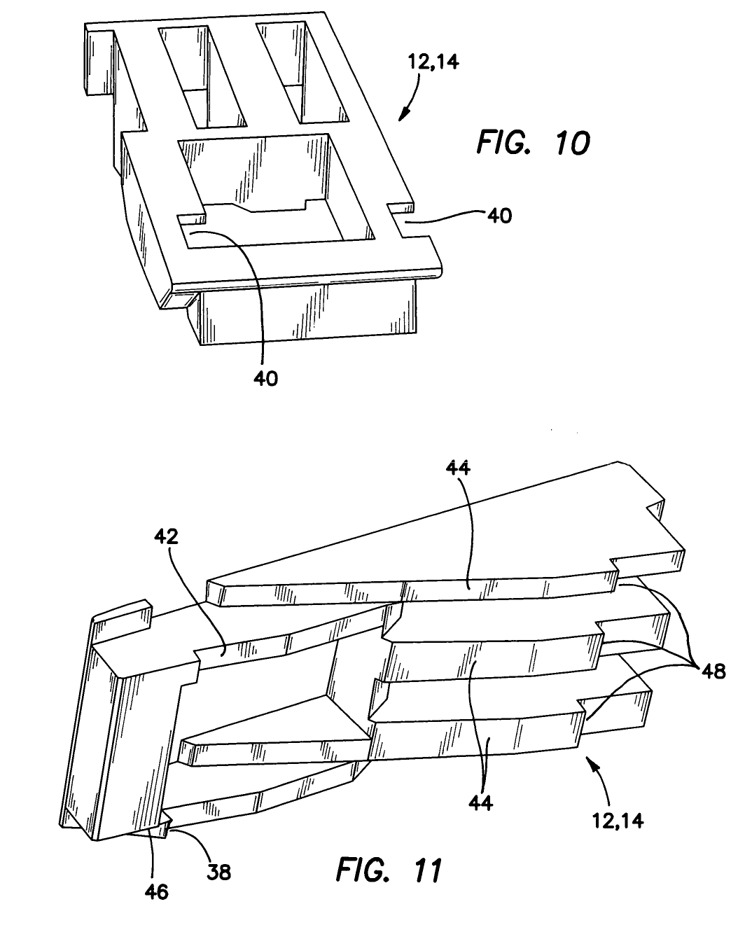

It should noted in view of FIGS. 1-11 that plunger 16 and half-cages 12 and 14 are open structures with a multiplicity of through holes and open interiors and sides wherever possible in a manner consistent with strength. The open-structure construction of plunger 16 and half-cages 12 and 14 allow for ready infiltration of tissue elements and healing or scarification agents to fill and consolidate the structure over time after fusion cage 10 is implanted in the spine. In this manner the spinal column will fuse at the implant site and securely encapsulate fusion cage 10.

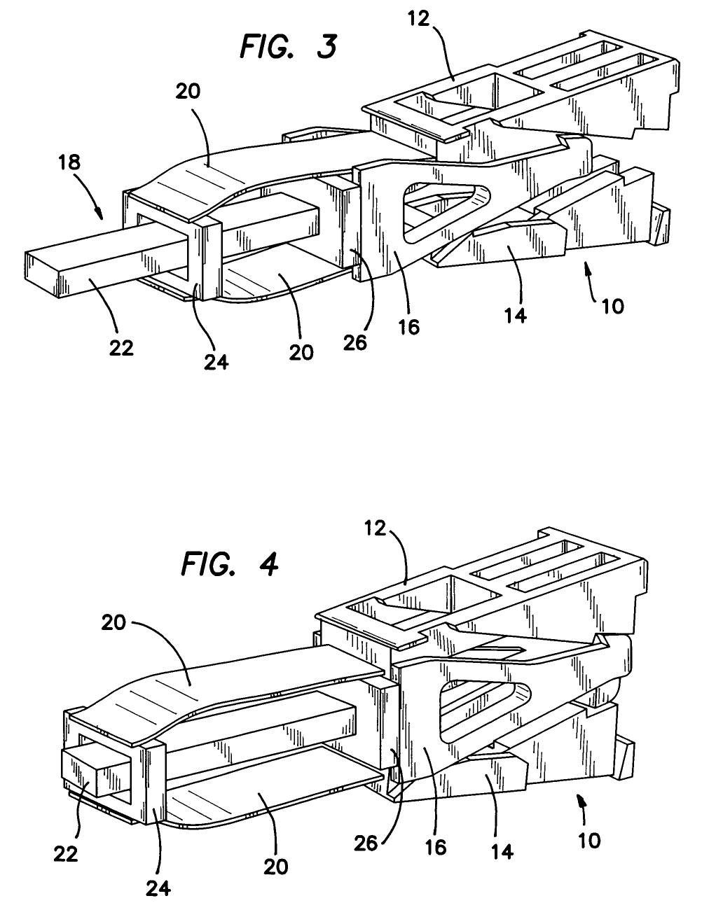

The invention having been described in its illustrated embodiment in connection with FIGS. 1-11 it can now be appreciated that fusion cage 10 has certain symmetries in its shape that provide the basis for its operation and for providing a predetermined lordosis. Half-cage 12 and 14 are characterized by a mirror symmetry with respect to each through the longitudinal axis when assembled. Viewing FIG. 7 it can be appreciated that surfaces 44 on three ribs 54 provide distal ramped surfaces for half-cage 12 and 14. As seen in FIGS. 7 and 11 surfaces 40 and 42 provide proximal ramped surfaces for half-cage 12 and 14. Plunger 16 as shown in FIG. 6 has a left side as viewed from its proximal end such that cam surface 46 will slide on ramp surface 44 of the left side of lower half-cage 14 as suggested in FIGS. 2 and 3, while cam surface 46 on the right side of plunger 16 slides on ramp surface 44 of the right side upper half-cage 12 as shown in FIGS. 2-5. Meanwhile ramped cam surface 56 on the lower right side plunger 16 as shown in FIG. 6 slides over surface 52 on the right side of lower half-cage 14 as shown in FIG. 3 as ramped cam surface 56 on the upper left side plunger 16 as shown in FIG. 6 slides over surface 52 on the left side of upper half-cage 12 as suggested in FIG. 3. When plunger 16 is fully inserted, the rear or proximal end 58 of plunger 16 slides off surface 52 and snaps into a locked position against proximal notches 40 in half-cage 12 and 14. At the same time cam surfaces 46 slide off surface 44 and snap into a locked position against distal notch 38 in half-cage 12 and 14. The inversion symmetry of the elements of fusion cage 10 thus provide symmetric functional operation during assembly of fusion cage 10 between the corresponding surfaces on the left side of upper half-cage 12 with the right side of lower half-cage 14, and the right side of upper half-cage 12 with the left side of lower half-cage 14.

The proximal ends of half-cages 12 and 14 are separated by the distance defining the height of the proximal end 58 of plunger 16, and distal ends of half-cages 12 and 14 are separated by the distance defining the height of the distal end 34 of plunger 16. The degree of lordosis is thus precisely define and maintained by the structural design and locking of plunger 16 between half-cages 12 and 14.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A spinal fusion cage comprising:
an upper half-cage;
a lower half-cage; and
a plunger with a cam, where the upper half-cage and lower half-cage have a first collapsed configuration which has a thin, flat, rectangular envelope and a second expanded configuration, the half-cages having at least one ramped surface on which the cam of the plunger rides, the cam bearing against the ramped surface and spreading the two half-cages apart.

2. The fusion cage of claim 1 where the plunger has two cams and where each half-cage has a ramped surface on which the cam of the plunger rides, the cam bearing against the ramped surface and spreading the two half-cages apart by a predetermined distance proximally and distally to define a predetermined lordosis.

3. The fusion cage of claim 1 where the plunger and half-cages each have an open structure to allow tissue infiltration therein.

4. The fusion cage of claim 1 where the plunger and half-cages further comprise a locking mechanism so that when the plunger is fully inserted between the half-cages, the plunger is locked into position.

5. The fusion cage of claim 4 where the half-cages have a proximal and distal end and where the locking mechanism locks the plunger between the half-cages at both the proximal and distal ends.

6. The fusion cage of claim 1 where the upper and lower half-cages have an identical shape and are arranged and configured to mesh with each other in the collapsed configuration.

7. The fusion cage of claim 1 where the half-cages each have a plurality of proximal ramps and a plurality of distal ramps, and where the plunger has a plurality of proximal cam surfaces arranged and configured to slide on the plurality of proximal ramps and a plurality of distal cam surfaces arranged and configured to slide on the plurality of distal ramps to spread the half-cages apart.

8. The fusion cage of claim 7 where the fusion cage has a longitudinal axis and where the plunger and half-cages are arranged and configured so that the operational combination of proximal cam surfaces with the proximal ramps and the operational combination of distal cam surfaces and distal ramps to spread the half-cages apart are inversely symmetric with respect to the longitudinal axis of the fusion cage.

9. The fusion cage of claim 8 where the plunger and half-cages are arranged and configured so that the inversely symmetric operational combination of the cam surfaces and ramps with respect to the longitudinal axis of the fusion cage provide during assembly of the fusion cage functional coaction between the corresponding surfaces on the left side of the upper half-cage with the right side of lower half-cage, and the right side of upper half-cage with the left side of lower half-cage.

10. The fusion cage of claim 1 where the proximal end of the plunger has a defined first height so that the corresponding proximal ends of the half-cages when the fusion cage is fully assembled are separated by the distance determined by the first height of the proximal end of the plunger, and where the distal end of the plunger has a defined second height so that the corresponding distal ends of the half-cages when the fusion cage is fully assembled are separated by the distance determined by the second height of the distal end of the plunger to provide a predetermined degree of lordosis.

11. The fusion cage of claim 1 where the half-cages further comprise flanges with notches defined therein and where the plunger is initially coupled to the proximal end of the two half-cages by means of engagement of the cam on the plunger with the notches in the flanges defined in the half-cages, when the plunger longitudinally extends from the two half-cages in the proximal direction.

12. A spinal fusion cage is comprised of:
an upper half-cage;
a lower half-cage; and
a plunger, where the upper half-cage has a distal portion which nests with the opposing distal portion of the lower half-cage so that the two half-cages provide a thin, flat, rectangular envelope, the half-cages have at least one inner ramped surface on which a cam of the plunger rides, the cam bearing against the ramped surface and distally spreading the two half-cages apart, at least one ramped surface on the proximal portion of the plunger bearing against an adjacent proximal interior surface of at least one of the half-cages so that at the same time as the plunger is being slid distally, the half-cages are being forced apart proximally.

13. A spinal fusion cage is comprised of:
an upper half-cage;
a lower half-cage; and
a plunger, where the upper half-cage has a distal portion which nests with the opposing distal portion of the lower half-cage so that the two half-cages provide a thin, flat, rectangular envelope, the half-cages have opposing inner ramped surfaces on which a top and bottom cam surface of the plunger rides, the cam bears against the ramped surfaces on the interior surfaces of the two half-cages and distally spreads them apart, two ramped surfaces on the proximal portion of the plunger bear against adjacent proximal interior surfaces of the half-cages so that at the same time as the plunger is being slid distally, the half-cages are forced apart proximally.

14. An assembly kit for a spinal fusion cage is comprised of:
a removable pusher tool;
an upper and lower half-cage having parallel longitudinal axes; and
a plunger which is slid between the half-cages by means of the removable pusher tool, where the half-cages are shaped in a complementary fashion so that when the upper half-cage is disposed on top of the lower half-cage, a distal portion of the lower and upper half-cages mesh with each other, so that the two half-cages assume the form of a collapsed fusion cage to provide a thin, flat, rectangular envelope with parallel upper and lower surfaces, the half-cages have opposing inner ramped surfaces on which a top and bottom cam surface of the plunger rides, as the plunger is forced distally between the two half-cages by the pusher tool, the cam which bears against the ramped surfaces on the interior surfaces of the two half-cages distally spreading the two half-cages apart in a direction perpendicular to their longitudinal axis, two symmetrically formed ramped surfaces on the proximal portion of the plunger bear against the proximal interior surfaces of the half-cages so that at the same time as the plunger is being slid distally towards a locked final position by the pusher tool, the half-cages are forced apart proximally, the ramps on the two half-cages and on the plunger being designed so that a desired degree of lordosis is obtained between the two-cages when in their final configuration.

15. The kit of claim 14 where the ramped interior surfaces have a mating and locking notch in the distal ends of the ramped interior surfaces of the two half-cages and where the plunger has a cam which moves into the mating and locking notch when the cam reaches the end of the ramped interior surfaces of the two half-cages, the half-cages then moving together to capture and lock the plunger between them.

16. The kit of claim 14 where the half-cages have flanges with a proximal notch defined therein, where the plunger has a distal cam and is initially coupled to the proximal end of the two half-cages by means of engagement of the distal cam on the plunger is fitted into the notches in the flanges defined in the half-cages, while the plunger longitudinally extends from the two half-cages in the proximal direction.

17. A method of deploying a spinal fusion cage comprising:
disposing in a spinal space an upper half-cage and lower half-cage in a first collapsed configuration which has a thin, flat, rectangular envelope and a second expanded configuration, the half-cages having at least one ramped surface on which the cam of the plunger rides; and
distally advancing a plunger between the upper half-cage and lower half-cage, the plunger having a cam bearing against the ramped surface and spreading the two half-cages apart.

18. The method of claim 17 where distally advancing the plunger comprises advancing a plunger with two cams and where each half-cage has a ramped surface on which the cam of the plunger rides, the cam bearing against the ramped surface and spreading the two half-cages apart by a predetermined distance proximally and distally to define a predetermined lordosis.

19. The method of claim 17 further comprising providing the plunger and half-cages each with an open structure to allow tissue infiltration therein.

20. The method of claim 17 further comprising locking the plunger into position between the half-cages when the plunger is fully inserted between the half-cages.

21. The method of claim 20 where locking the plunger comprises locking the plunger between the half-cages at both the proximal and distal ends of the half-cages.

22. The method of claim 17 where disposing the upper half-cage and lower half-cage in a first collapsed configuration comprises disposing identically shaped upper and lower half-cages in an intermeshed relationship with each other in the collapsed configuration.

23. The method of claim 17 where distally advancing the plunger comprises sliding a plurality of proximal cam surfaces on the plunger on a plurality of proximal ramps and sliding a plurality of distal cam surfaces on the plurality of distal ramps to spread the half-cages apart.

24. The method of claim 23 where sliding a plurality of proximal cam surfaces on the plunger on a plurality of proximal ramps and sliding a plurality of distal cam surfaces on the plurality of distal ramps to spread the half-cages apart comprises operationally combining the proximal cam surfaces with the proximal ramps and the distal cam surfaces with distal ramps in a manner inversely symmetric with respect to the longitudinal axis of the fusion cage.

25. The method of claim 24 where operationally combining the proximal cam surfaces with the proximal ramps and the distal cam surfaces with distal ramps in a manner inversely symmetric with respect to the longitudinal axis of the fusion cage comprises assembling the fusion cage by using the corresponding surfaces on the left side of the upper half-cage with the right side of lower half-cage, and the right side of upper half-cage with the left side of lower half-cage.

26. The method of claim 17 where the half-cages have proximal and distal ends and where distally advancing the plunger comprises spreading the proximal ends of the half-cages by inserting therebetween a proximal end of the plunger which has a defined first height, and spreading the distal ends of the half-cages when the fusion cage by inserting therebetween the distal end of the plunger which has a defined second height so that when the fusion cage is fully assembled a predetermined degree of lordosis is provided.

27. The method of claim 17 further comprising initially coupling the plunger into proximal notches defined in flanges of the half-cages by means of engagement of the cam on the plunger, when the plunger longitudinally extends from the two half-cages in a proximal direction.

28. The method of claim 17 further comprising coupling a removable pusher tool to the plunger prior to distally advancing the plunger, automatically disengaging the pusher tool from the plunger after the plunger has been distally advanced beyond a predetermined distance between the half-cages, locking the plunger to the half-cages by distally advancing the tool, and removing the pusher tool.

29. The method of claim 28 where locking the plunger comprises engaging a cam on the plunger with a mating and locking notch in the distal ends of ramped interior surfaces of the two half-cages.

30. The method of claim 28 where locking the plunger comprises engaging the plunger with a mating and locking notch in the proximal ends of the two half-cages.

31. The method of claim 29 where locking the plunger comprises engaging the plunger with a mating and locking notch in the proximal ends of the two half-cages.
